(19) United States
(12) Patent Application Publication
(10) Pub. No.: US 2026/0007853 A1
(43) Pub. Date: Jan. 8, 2026

(54) EXTENDED REALITY DEVICE FOR REAL-TIME BIOMETRIC CLASSIFICATION AND IMMERSIVE THERAPEUTIC INTERVENTION

(71) Applicant: OpiAid, Inc., Wilmington, NC (US)

(72) Inventors: Dominic Cincotti, Atlanta, GA (US); David Reeser, Wilmington, NC (US)

(21) Appl. No.: 19/257,753

(22) Filed: Jul. 2, 2025

Related U.S. Application Data
(60) Provisional application No. 63/666,877, filed on Jul. 2, 2024.

(51) Int. Cl. A61M 21/02; A61M 21/00; G06F 3/01; G06T 19/00; G16H 20/70; G16H 40/67

(52) U.S. Cl. CPC A61M 21/02; G06F 3/012; G06F 3/013; G06T 19/00; G16H 20/70; G16H 40/67; A61M 2021/005; A61M 2205/3313; A61M 2205/6009; A61M 2209/088; A61M 2230/04

(57) ABSTRACT

A self-contained extended reality (XR) device is disclosed for real-time detection and mitigation of substance use disorder symptoms. The headset includes integrated biometric sensors for capturing physiological signals such as pupil dilation, heart rate, and head movement. A locally stored machine-learned model analyzes these signals to classify cognitive or affective states indicative of withdrawal, craving, or relapse risk. Upon detecting a threshold state, the headset automatically renders an immersive therapeutic environment selected to mitigate the user's condition. Interventions may include spatialized audio, visual modulation, and avatar-guided interactions. The device further supports adaptive personalization, secure local data storage, and optional transmission of session summaries to authorized care providers. The system operates without external computing devices and is optimized for privacy-preserving, user-specific behavioral health support.

### CROSS-REFERENCE TO RELATED APPLICATIONS

This application claims the benefit of U.S. Provisional Patent Application No. 63/666,877 entitled “METHODS, DEVICES, AND SYSTEMS FOR PROVIDING MEDICATION ASSISTED SPATIAL TREATMENTS” filed on Jul. 2, 2024, the entire content of which is incorporated by reference herein.

### TECHNICAL FIELD

The present invention relates generally to extended reality (XR) systems, biometric signal processing, and therapeutic interface technologies. More specifically, the invention relates to an XR device configured to acquire physiological data, perform real-time classification of cognitive or affective states, and initiate immersive interventions based on such classifications.

### BACKGROUND

Extended Reality (XR) devices have gained widespread use in entertainment, training, and education, yet their application in real-time behavioral health interventions remains limited. While some systems support basic visualization or guided experiences, they typically lack the capacity to adapt in real time based on biometric signals reflecting a user's physiological or cognitive state.

Simultaneously, wearable devices capable of tracking heart rate, pupil dilation, or motion have become increasingly accessible. However, these biometric inputs are often processed in isolation and disconnected from immersive platforms that could deliver targeted interventions. Current solutions do not leverage sensor fusion and machine learning within a standalone XR device, such as a headset, to identify withdrawal symptoms or escalate therapeutic interventions for substance use disorder (SUD).

Moreover, many digital health tools require smartphone pairing or external processing, introducing latency, fragmented experiences, and reliance on user input. There is a need for a self-contained, adaptive XR device that continuously monitors biometric signals, classifies psychological states in real time, and renders immersive environments to mitigate relapse risk or withdrawal distress.

In addition, existing systems fail to offer robust mechanisms for securing and managing the sensitive biometric and behavioral data generated during therapeutic sessions. There remains a critical gap in providing encrypted local storage, access logging, and secure transmission protocols to ensure that real-time classification outputs and intervention metadata can be selectively shared with authorized clinicians or care teams without compromising patient privacy or violating consent boundaries. A trusted, device-integrated framework for secure biometric data management and optional transmission is necessary for clinical validation, longitudinal tracking, and regulatory compliance.

Accordingly, there is a need for improved methods, devices, and systems for real-time biometric state detection, immersive therapeutic intervention, and secure management of physiological and behavioral data within a standalone extended reality (XR) device.

### SUMMARY

In one embodiment, the system comprises an extended reality (XR) device configured to monitor biometric signals, classify user states, and deliver immersive therapeutic interventions. The headset includes a head-mounted display capable of rendering stereoscopic visual environments and one or more biometric sensors integrated into the housing. The biometric sensors may include eye-tracking cameras, photoplethysmography (PPG) modules, and inertial measurement units (IMUs) for capturing gaze, heart rate, and head motion, respectively.

In certain aspects, the headset includes a processor and memory unit operatively coupled to the sensors and display. The memory stores a machine-learned classification model trained to identify psychological states associated with substance use disorder (SUD), such as withdrawal, craving, or emotional dysregulation. The processor is configured to collect and preprocess biometric data in real time, apply the classification model, and generate a severity score indicating the likelihood or intensity of a high-risk state.

If the severity score exceeds a configurable threshold, the headset automatically initiates rendering of an immersive therapeutic environment. In one embodiment, the environment may include spatialized audio, ambient visual effects, or guided avatar interactions. These environments are selected from a local library and may be prioritized based on prior response efficacy, user preference, or longitudinal biometric trends.

In some embodiments, the headset includes logic for smoothing biometric data over time, enforcing minimum intervention durations, and adjusting environment parameters based on real-time signal feedback. For example, if the user's distress signals remain elevated, the system may escalate the intervention to a more structured or interactive format.

Personalization is supported through a baseline calibration routine, user-configurable settings, and optional clinician-defined thresholds. The system may store session metadata (including classification outputs and intervention identifiers) in encrypted local memory. Where authorized, summary metrics may be securely transmitted to remote care teams via a wireless communication interface.

The headset operates independently of external computing devices and supports both offline and network-enabled modes. It is designed for deployment in clinical, residential, or ambulatory environments, and provides privacy-preserving, real-time behavioral health support through a single integrated device.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The present embodiments are illustrated by way of example and are not intended to be limited by the figures of the accompanying drawings. In the drawings:

FIG. 1 is a block diagram of an XR device configured with integrated biometric sensors, a classification engine, and an immersive display for therapeutic intervention.

FIG. 2 is a flowchart illustrating the signal processing and classification pipeline performed by the XR device to detect withdrawal severity.

FIG. 3 is a schematic illustration of an immersive therapeutic environment rendered in response to a classified biometric state.

FIG. 4 is a diagram showing components of a multi-device ecosystem including immersive devices, biometric wearable devices, and their use by both patient and clinician users.

FIG. 5 is a timeline showing real-time biometric signal capture, classification intervals, and corresponding intervention events during a sample therapeutic session.

FIG. 6 is a schematic illustration of a consent management interface showing quaternion access controls and patient consent options for data sharing with authorized clinicians.

FIG. 7 is a diagram depicting a secure sphere transfer system for multi-tiered data access between patient and clinician users, showing various layers of biometric and clinical data.

### DETAILED DESCRIPTION

Systems disclosed herein may include various immersive devices, such as AR/VR/XR headsets, smart glasses, and holographic displays, as well as various biometric wearable devices, such as smart rings, smartwatches, health and fitness tracking bands, and spatial earbuds. While the embodiment discussed in greater detail below focuses on the features of a specific XR device—an XR head-worn device or XR headset—to illustrate aspects of the subject matter disclosed herein, it is appreciated that the subject matter disclosed herein is not limited to the specific XR headset discussed below.

### Overview of the XR Device Architecture

### 1.1. Head-Mounted Display Components

In one embodiment, the disclosed system comprises a head-mounted extended reality (XR) device configured for real-time biometric classification and therapeutic intervention. The XR device is self-contained and includes integrated sensing, processing, display, and storage components, enabling continuous operation without requiring external computing or smartphone tethering.

The headset includes a pair of stereoscopic (or alternatively monoscopic) display(s) positioned in front of the user's eyes. These displays are configured to render immersive visual environments with high-resolution graphics, optionally incorporating dynamic lighting, ambient animations, and avatar-driven scenarios. The displays may support variable refresh rates and high dynamic range rendering to accommodate different therapeutic contexts.

### 1.2. Integrated Biometric Sensors

Integrated within the headset housing are one or more biometric sensors. These sensors may include infrared cameras for pupil tracking and eye movement detection, photoplethysmography (PPG) sensors for pulse waveform and heart rate monitoring, and inertial measurement units (IMUs) for capturing head movement and motion dynamics. In certain embodiments, the biometric sensor suite also includes proximity sensors, temperature sensors, and capacitive or photonic elements for additional physiological measurements.

In further embodiments, the biometric sensor suite may comprise additional modalities to support comprehensive physiological and behavioral state monitoring. The headset may incorporate electroencephalography (EEG) sensors integrated within the headband or housing, configured for non-invasive measurement of neurological activity. These EEG sensors may provide up to eight channels of data, with sampling rates up to 1000 Hz, supporting detection of cognitive and affective state transitions relevant to substance use disorder management.

Galvanic skin response (GSR) sensors may be included at forehead, temporal, or other contact points to monitor sympathetic nervous system activity, with microsiemens-level resolution for accurate assessment of arousal or stress.

Environmental context sensors, such as ambient light, temperature, humidity, and acoustic monitors, may be integrated to capture external conditions potentially impacting physiological states or device operation. The sensor suite may also comprise micro-vibration detectors configured for tremor analysis and agitation monitoring, with sub-millimeter displacement sensitivity, as well as proximity sensors for verifying device positioning and contact quality.

Each biometric sensor modality may be designed to operate at a sampling rate optimized for its specific signal characteristics, for example, infrared cameras for eye tracking at up to 1000 Hz, photoplethysmography (PPG) sensors at up to 500 Hz, and inertial measurement units (IMUs) at up to 1000 Hz. Signal-to-noise ratios, resolution, and artifact rejection capabilities may be selected to meet or exceed professional-grade standards for continuous behavioral health monitoring.

In some embodiments, the headset may include cross-modal validation protocols or sensor fusion logic implemented in hardware or firmware. These may synchronize multi-modal data streams using hardware-based timestamping with nanosecond precision, advanced interpolation techniques to align signals of differing temporal characteristics, and anomaly detection logic to identify corrupted or tampered signals through statistical correlation or machine learning-based quality assessment.

### 1.3. On-Device Processor and Memory

The device includes a processor, such as a system-on-chip (SoC), capable of executing machine-learned models and signal processing logic. The processor is operatively coupled to an onboard memory unit that stores biometric data buffers, classification models, therapeutic environment definitions, and configuration parameters. The memory may include a combination of volatile (e.g., RAM) and non-volatile (e.g., flash) components.

### 1.4. Wireless Connectivity and Power Supply

The headset further includes wireless radios configured for local or wide-area communication. In some embodiments, the headset supports Wi-Fi and Bluetooth® protocols, allowing optional secure transmission of session summaries or intervention metadata to a remote server. Power is supplied by an internal rechargeable battery, and the housing may incorporate thermal regulation and tamper-detection elements for safety and compliance.

The components of the XR device are arranged to support continuous biometric monitoring, low-latency signal classification, and responsive environment rendering without requiring input from external devices. The user interface may include gaze-based selection, voice commands, or gesture recognition, allowing hands-free operation of therapeutic functions.

### 1.5. Professional Integration Hardware Modules

In certain embodiments, the XR device may further comprise a dedicated professional integration module implemented as a hardware or firmware subsystem. This module may include one or more secure communication processors configured to enable authenticated connectivity with external clinical information systems or professional devices. The hardware integration may support compliance with healthcare interoperability standards, including but not limited to FHIR (Fast Healthcare Interoperability Resources) and HL7 (Health Level Seven) protocols, by providing hardware-accelerated encryption, key management, and secure data routing.

The professional integration hardware module may further incorporate tamper-resistant memory for secure credential storage, cryptographic accelerators for encrypted data exchange, and dedicated interfaces for connecting to external authentication hardware or clinical monitoring equipment. The module may implement role-based access control at the hardware level, wherein hardware-enforced permission registers govern the availability of certain device features or data channels based on authenticated professional credentials.

In certain embodiments, a multi-tier notification controller may be implemented as a dedicated logic block, configured to trigger secure professional alerts or escalation sequences in response to detection of critical physiological events. This controller may be operatively coupled to the primary processing unit and secure communication subsystem, ensuring that real-time professional notification is executed with minimal latency and hardware-backed security. Hardware-based audit logging functions may also be included to generate immutable records of all professional access and device interactions for compliance verification.

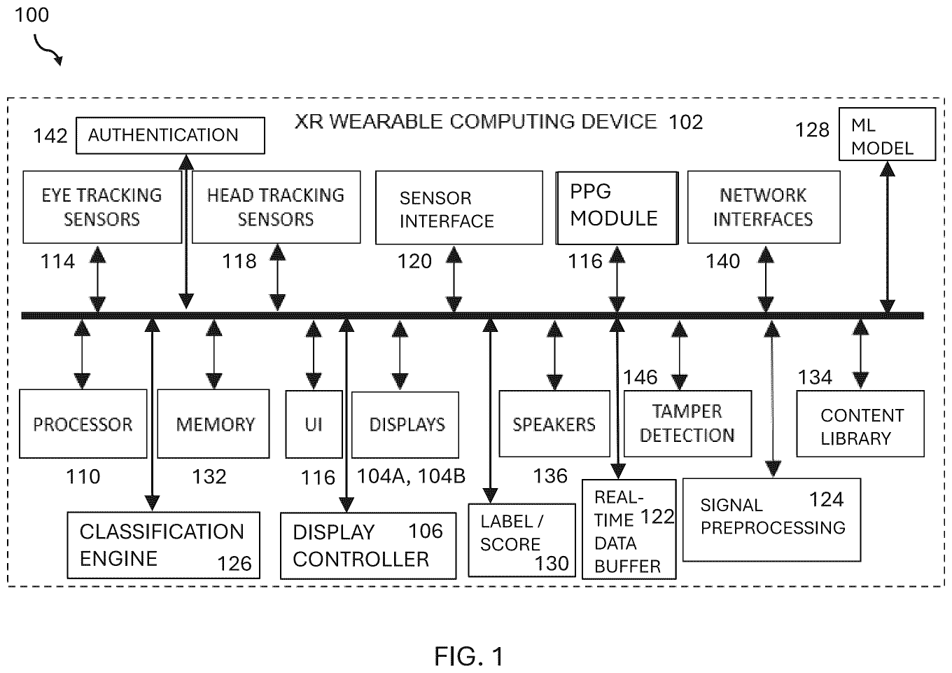

FIG. 1 illustrates a block diagram of an extended reality (XR) device system 100 configured for real-time biometric classification and immersive therapeutic intervention. The XR device 102 is shown as comprising multiple integrated modules and subsystems, each operatively coupled to support continuous behavioral health monitoring and intervention.

The headset 102 includes a pair of stereoscopic displays 104A, 104B positioned to render immersive visual content to the user. These displays may be configured to support high-resolution, wide field-of-view rendering, optionally incorporating dynamic lighting, ambient visual effects, and three-dimensional avatar-driven scenarios. The displays are controlled by a display controller module 106, which is in turn coupled to a central processing unit (CPU) 110.

Integrated within the housing of the headset 102 are a plurality of biometric sensors. In one embodiment, the biometric sensors may include one or more infrared cameras 114 for eye tracking and pupil dilation measurement, a photoplethysmography (PPG) module 116 for pulse waveform and heart rate detection, and an inertial measurement unit (IMU) 118 comprising a gyroscope and accelerometer for detecting head motion. Additional sensors, such as proximity sensors, temperature sensors, or capacitive elements, may also be included to extend physiological measurement capabilities.

The biometric sensors are operatively coupled to a sensor interface module 120, which performs initial signal conditioning and forwards the acquired data to the main processing subsystem. The sensor interface module 120 is connected to a real-time data buffer 122, which implements rolling window storage for each modality, allowing for temporal analysis and trend detection.

The central processing unit (CPU) 110 is further configured to execute signal preprocessing logic 124. This module may perform noise reduction, normalization, temporal filtering, and artifact rejection for each biometric signal channel. Preprocessed signals are synchronized and routed to a classification engine 126 implemented in software or hardware, such as a neural network accelerator.

The classification engine 126 accesses a locally stored machine-learned model 128. The model 128 is trained to identify psychological or affective states associated with substance use disorder (SUD), including withdrawal onset, craving episodes, and elevated relapse risk. Based on the synchronized, preprocessed signals, the classification engine 126 outputs one or more state labels or a withdrawal severity score 130, which is compared to one or more predetermined thresholds stored in memory 132.

When the severity score 130 exceeds a configurable threshold, the CPU 110 initiates a therapeutic intervention by selecting an appropriate immersive environment from a content library 134. The immersive environment parameters, such as spatialized audio cues, visual modulation, and avatar guidance, are rendered in real time through the displays 104A, 104B and integrated speakers/spatial or immersive audio device 136. The system supports dynamic adaptation of environment features based on feedback from ongoing biometric monitoring.

A local memory unit 132 is provided for the storage of signal buffers, classification outputs, environment configuration data, and intervention event logs. The memory unit 132 comprises both volatile and non-volatile components and supports encrypted storage partitions to preserve user privacy.

The XR device 102 further comprises a wireless communication interface 140, which may support Wi-Fi and Bluetooth® protocols. This interface enables the optional transmission of de-identified session summaries and intervention metadata to remote servers or authorized care team dashboards, subject to user consent and privacy safeguards.

The block diagram of FIG. 1 may further depict auxiliary modules such as a user authentication module 142 for secure access and tamper detection circuitry 146 to enforce device security and compliance.

The arrangement of components as depicted in FIG. 1 enables a self-contained, adaptive XR device capable of continuous biometric monitoring, on-device state classification, and responsive therapeutic intervention without requiring connection to external computing devices. All signal processing, classification, and environment rendering are performed locally, with privacy-preserving data management and configurable sharing options as described.

### Biometric Signal Acquisition and Preprocessing

2.1. Signal Sampling from Multiple Sensor Modalities

The XR device continuously acquires physiological data from a plurality of onboard biometric sensors. These sensors may operate in parallel and capture multimodal input streams including, but not limited to, pupil dilation (via infrared eye-tracking cameras), pulse waveform data (via PPG sensors), and inertial motion patterns (via gyroscopes and accelerometers). Each sensor channel operates with its own temporal sampling rate and resolution, which may be predefined or adjusted based on user configuration, environmental context, or system performance constraints.

In some embodiments, the XR device may employ sensor fusion algorithms configured to synchronize data streams from heterogeneous sensor modalities. Sensor fusion may be implemented in hardware, firmware, or a combination thereof, utilizing dedicated timestamping circuits to assign high-precision temporal markers to each data sample. Timestamp resolution may be sufficient to achieve nanosecond-level accuracy, enabling effective alignment of signals with differing native sampling rates and temporal characteristics.

To harmonize and integrate multimodal data, the system may apply interpolation techniques and cross-modal signal alignment protocols. These protocols may be designed to correct for drift, latency, or phase offsets between sensor channels. In some embodiments, signal fusion logic may validate temporal consistency among sensor readings and may detect anomalies, corruption, or potential tampering by applying statistical correlation or machine learning-based quality assessment algorithms.

Cross-modal validation protocols may be used to identify inconsistencies in the acquired data, such as artifacts arising from sensor malfunction, user movement, or environmental interference. Upon detection of invalid or suspect segments, the affected data may be flagged for exclusion from downstream classification or intervention processing.

### 2.2. Noise Reduction and Signal Normalization

Raw sensor data streams are preprocessed locally within the headset to improve signal quality and facilitate accurate classification. Preprocessing may include temporal filtering, artifact rejection (e.g., removal of blink-related spikes from eye-tracking data), and normalization techniques such as min-max scaling or z-score standardization. These operations are tailored to each biometric channel and are optimized for real-time computation to avoid latency in the subsequent classification pipeline.

In some embodiments, preprocessing steps also include interpolation or resampling to harmonize signal lengths or align data across modalities. The system may apply heuristics to identify invalid or corrupted data segments and exclude them from classification input.

The preprocessing logic may further incorporate cross-sensor validation, wherein data from multiple modalities are compared to identify discordant readings or corrupted samples. When signal segments are determined to be inconsistent or to fall outside of expected statistical norms, such segments may be excluded from feature vector construction or downstream analysis, thereby reducing the risk of misclassification due to faulty input.

### 2.3. Rolling Buffer and Time Windowing

To enable real-time analysis while preserving short-term trends, the XR device maintains a rolling buffer for each biometric modality. This buffer stores a fixed-duration window of preprocessed data, such as the most recent 5-10 seconds of continuous measurements. The rolling buffers are updated at defined intervals (e.g., every 500 milliseconds), and the aligned data from each buffer is treated as a synchronized snapshot for input into the classification model.

This time-windowed structure allows the system to maintain responsiveness to sudden physiological changes while also providing sufficient temporal context to identify evolving states such as increasing distress, relapse risk, or withdrawal onset.

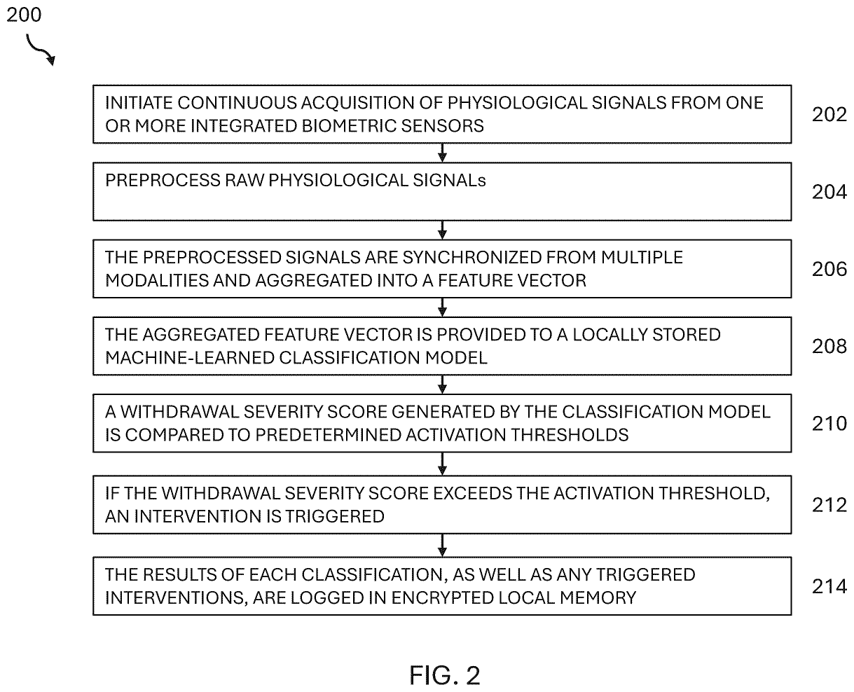

FIG. 2 presents a flowchart depicting the signal processing and classification pipeline 200 executed by the XR device to detect withdrawal severity and initiate corresponding therapeutic interventions. The illustrated sequence delineates discrete functional stages, each of which may be implemented in hardware, software, or a combination thereof, within the XR device.

At step 202, the pipeline is initiated by continuous acquisition of physiological signals from one or more integrated biometric sensors. Such sensors may include, but are not limited to, infrared cameras for eye tracking, photoplethysmography (PPG) modules for heart rate detection, and inertial measurement units (IMUs) for head motion analysis. Signal streams from each modality are sampled at respective, predefined rates and forwarded to the subsequent processing stage.

At step 204, the raw physiological signals are subjected to preprocessing. This preprocessing may include temporal filtering to reduce noise, normalization to standardize measurement scales, and artifact rejection to remove spurious signal components such as blink-related noise or motion artifacts. The preprocessed signals are synchronized and buffered in rolling data windows to facilitate temporal analysis.

At step 206, the synchronized and preprocessed signals from multiple modalities are aggregated into a feature vector, which may capture instantaneous physiological states as well as short-term trends. This feature vector is prepared as input to the on-device classification engine.

At step 208, the aggregated feature vector is provided to a locally stored machine-learned classification model. The model may comprise a neural network architecture optimized for edge execution, such as a convolutional or recurrent neural network. The classification model processes the input and produces one or more outputs, including a withdrawal severity score and, optionally, categorical state labels (for example, “stable,” “at risk,” or “acute withdrawal”).

At step 210, the withdrawal severity score generated by the classification model is compared to one or more predetermined activation thresholds. These thresholds may be stored in device memory and may be user-configurable or set by a clinician.

At step 212, if the withdrawal severity score exceeds the activation threshold, an intervention is triggered. The headset proceeds to select and render an immersive therapeutic environment, as described in FIG. 1, tailored to the classified state. If the threshold is not met, the system continues monitoring and analysis without initiating an intervention.

At step 214, the results of each classification, as well as any triggered interventions, are logged in encrypted local memory. These logs may include timestamps, summary statistics, environment identifiers, and relevant biometric signal features, and may be used for future trend analysis or optional reporting to authorized care providers.

The depicted flowchart enables closed-loop, real-time behavioral health support by continuously acquiring physiological signals, classifying risk states, and responsively delivering interventions, all within a self-contained XR device. The signal processing and classification pipeline may be configured to operate with low latency and minimal external dependencies, ensuring robustness in ambulatory, residential, or clinical deployment scenarios.

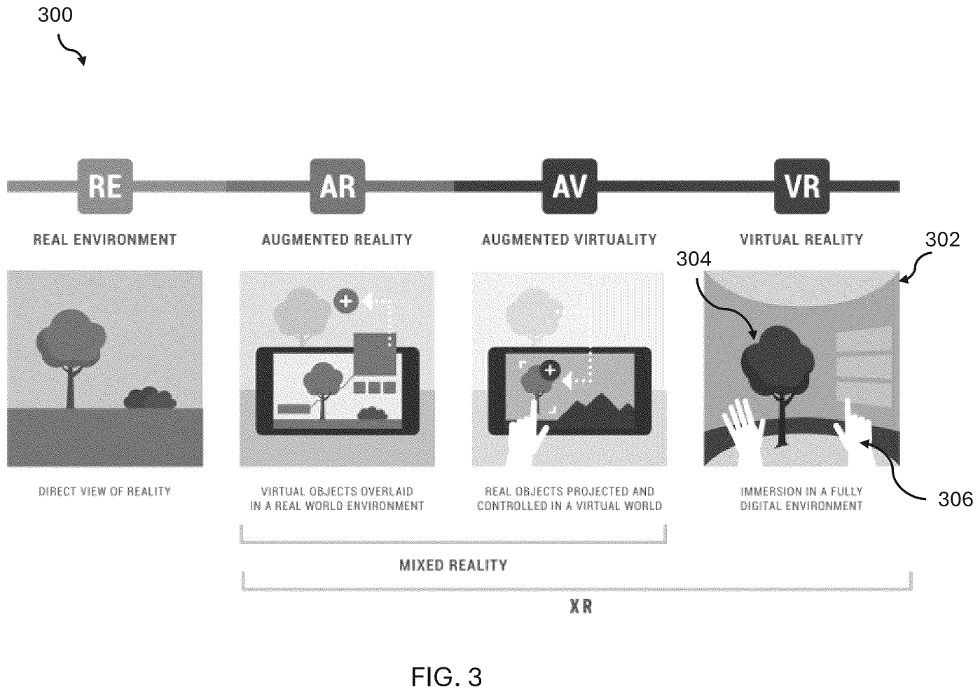

FIG. 3 depicts a schematic illustration of an immersive therapeutic environment 300 generated and rendered by the XR device in response to detection of a classified biometric state, such as acute withdrawal or elevated relapse risk. The diagram is intended to exemplify how the system adapts intervention content in real time based on user-specific physiological indicators.

Within the illustrated environment 300, a virtual scene 302 is rendered through the headset's stereoscopic displays. The virtual scene 302 comprises a three-dimensional, immersive landscape, which may include natural settings such as forests, beaches, or tranquil indoor spaces. This scene 302 is rendered with high-resolution graphics and may be selected according to user preference, clinical recommendation, or historical intervention efficacy. In one embodiment, the scene 302 may be selected from a locally stored environment library.

Integrated within the environment 300 are spatial audio and visual effects 304. These effects may include dynamic lighting, ambient soundscapes, smooth color transitions, and subtle motion elements. Both audio and visual parameters are adaptively modulated in real time, based on ongoing biometric feedback. For example, spatial audio may include sounds such as flowing water or gentle wind, while visual effects may be adjusted to create a calming atmosphere when physiological distress is detected.

The ambient visual effects 304 may include dynamic lighting, gradual color transitions, and soft motion patterns, each of which may be modulated in response to ongoing biometric feedback. Spatialized audio sources 306 are positioned within the virtual scene, generating immersive soundscapes that may include flowing water, wind, or synthesized harmonics. The spatial audio is rendered using the headset's integrated speakers or paired audio devices and is dynamically adjusted to promote autonomic regulation and support the desired therapeutic effect.

In certain embodiments, a virtual avatar 306 may also be instantiated within the environment 300. The avatar 306 is configured to deliver guided prompts, verbal affirmations, or interactive exercises synchronized to the classified state and biometric trends of the user. For instance, the avatar may guide the user through relaxation or breathing exercises, adjusting gesture and speech cadence in response to real-time physiological data.

The therapeutic environment 300, as depicted in FIG. 3, is dynamically adapted according to the user's current state and biometric responses. Parameters governing spatial audio, visual effects, and avatar behavior may be escalated or de-escalated based on ongoing measurements. All relevant session metadata, including environment selection, intervention duration, and biometric recovery, are securely logged to support future personalization and therapeutic review.

### Classification of Withdrawal and Relapse-Associated States

### 3.1. Stored Machine-Learned Model Architecture

The XR device includes a locally stored machine-learned classification model configured to detect cognitive and affective states associated with substance use disorder (SUD), such as withdrawal onset, craving episodes, emotional dysregulation, or high relapse risk. In one embodiment, the model is a lightweight neural network optimized for edge execution. Architectures may include convolutional neural networks (CNNs), recurrent networks (RNNs), or hybrid models combining temporal and spatial feature extraction layers. The model is stored in non-volatile memory and executed on the device processor without the need for cloud inference.

### 3.2. Mapping Signal Features to Psychological States

The classification model receives synchronized, preprocessed biometric signal windows as input and maps these inputs to latent state representations. For example, a window showing pupil constriction, increased blink frequency, elevated heart rate, and head jitter may be classified as indicative of acute anxiety or early withdrawal. The model outputs one or more classification labels or scores representing predicted psychological states relevant to SUD management. These labels may be discrete (e.g., “stable,” “moderate risk,” “acute withdrawal”) or continuous (e.g., numeric severity values on a normalized scale).

Feature importance may be dynamically weighted by the model based on signal quality, user history, or time-of-day priors. The headset may also apply internal heuristics to verify that input data is sufficient for a valid classification decision before triggering any downstream response.

### 3.3. Scoring Logic and Threshold Comparisons

In some embodiments, the classification model produces a withdrawal severity score normalized to a fixed range, such as 0.0 to 1.0. This score is evaluated against a predetermined threshold to determine whether an immersive intervention should be initiated. Thresholds may be fixed by default or configured based on user preferences, clinical recommendations, or adaptive learning.

For instance, if the severity score exceeds a defined threshold (e.g., 0.7), the headset may proceed to initiate an intervention. Conversely, if the score remains below the threshold for a sustained period, the system may withhold or defer any response. Threshold-based logic ensures that interventions are not triggered by transient noise or benign fluctuations in biometric state.

### 3.4. Trend Smoothing and State Persistence Handling

To enhance classification stability, the system may apply temporal smoothing or decision hysteresis across successive severity scores. For example, three consecutive scores above a trigger threshold may be required before activating a therapeutic environment. Similarly, intervention suspension may require the score to remain below a reset threshold for a minimum duration (e.g., 30 seconds) to prevent premature interruption.

The system may also track state persistence and trajectory (e.g., a steadily rising score trend) to anticipate risk escalation even if absolute thresholds are not yet crossed. These logic layers enable more refined, patient-tailored activation criteria that balance responsiveness with reliability.

### 3.5 Temporal Pattern Analysis and Predictive Intervention Logic

In certain embodiments, the XR device may incorporate a temporal analysis engine configured to analyze time-sequenced biometric data and predict transitions in physiological or psychological state. The temporal analysis engine may be implemented using advanced neural network architectures, such as recurrent neural networks (RNNs), long short-term memory (LSTM) networks, convolutional neural networks (CNNs) for spatial-temporal feature extraction, or Transformer-based models capable of multi-modal temporal attention. These models may be executed on dedicated tensor processing units or optimized processor cores within the device, supporting real-time inference with minimal latency.

The system may analyze rolling windows of biometric data, such as up to 30 days of recorded measurements, to identify evolving trends and latent temporal dependencies that precede high-risk states. Correlation latency in such analysis may be maintained below 10 milliseconds, enabling timely prediction and proactive initiation of therapeutic interventions before acute episodes fully manifest. Baseline establishment protocols may incorporate individual variation, circadian rhythm analysis, medication effect compensation, and environmental context, allowing classification thresholds and predictive models to adapt to the user's unique patterns over time.

In some embodiments, the device may operate in a learning mode or an operational mode. During learning mode, the system may undergo supervised training phases, optionally with professional annotation of physiological events, validation periods with semi-supervised feedback, and controlled testing to assess efficacy metrics. Transition to operational mode may occur only after validation thresholds are achieved, at which point the system autonomously delivers interventions, monitors effectiveness, and continues to adapt classification parameters based on ongoing outcomes. The transition protocol may require professional or administrative review, ensuring clinical safety and accuracy.

Predictive intervention logic may utilize reinforcement learning, Bayesian inference, or ensemble modeling approaches to optimize intervention timing, selection, and intensity. The reward structure for learning may consider physiological recovery rates, professional review outcomes, user-reported efficacy, and long-term therapeutic progress. The system may apply multi-objective optimization to balance therapeutic impact, user comfort, and resource utilization, and to ensure that high-intensity or frequent interventions are appropriately reviewed by authorized professionals.

Predictive intervention algorithms may be configured to utilize reinforcement learning, Bayesian inference, and ensemble modeling techniques to optimize the selection and timing of therapeutic interventions. Multi-objective optimization criteria may balance intervention efficacy, user comfort, and resource utilization. Continuous validation of algorithm performance may be supported through professional review and adaptive tuning of model parameters in response to clinical outcomes.

### 3.6 Neural Network and Edge Processing Architecture

In some embodiments, the XR device may implement neural network architectures optimized for multi-modal biometric analysis and real-time therapeutic intervention. Neural network models may include convolutional neural networks (CNNs) for spatial biometric pattern recognition, long short-term memory (LSTM) networks for sequential and temporal pattern analysis, and Transformer architectures for multi-modal attention and feature correlation. These models may be deployed on dedicated tensor processing units or neural accelerators integrated within the device.

The neural network models may be optimized for edge deployment using techniques such as network quantization, pruning, and knowledge distillation, thereby reducing memory footprint and computation requirements without compromising accuracy. Hardware acceleration may be provided by parallel processing architectures, matrix multiplication optimizations, and on-chip memory hierarchies designed to minimize data transfer latency and support simultaneous multi-modal analysis.

In some embodiments, the system may further support federated learning protocols, enabling local model adaptation and collaborative training across multiple devices while preserving data privacy. Differential privacy mechanisms may be employed to ensure that updates to shared models do not expose individual user data. Secure aggregation techniques, such as homomorphic encryption, may be used to combine local model updates across devices before global model distribution, maintaining statistical robustness while safeguarding personal biometric information.

The system may also support synthetic data generation to supplement local training datasets, thereby improving model generalizability and robustness. All model updates, training events, and associated metadata may be versioned, authenticated, and logged for quality assurance, regulatory compliance, and potential rollback in the event of performance regressions.

In some embodiments, federated learning protocols may be supported, enabling distributed model training and adaptation across multiple devices while maintaining the privacy of locally stored biometric data. Synthetic data generation algorithms may further augment training datasets, enhancing model robustness without compromising user privacy. All model updates may be authenticated, versioned, and subject to audit for regulatory compliance.

### Immersive Therapeutic Intervention Logic

### 4.1. Library of Immersive Environments

The XR device stores a predefined library of immersive therapeutic environments designed to support users during episodes of withdrawal, craving, or emotional distress-including direct connection to medical professionals. Each environment comprises a structured combination of visual, auditory, and interactive elements aimed at eliciting calming, grounding, or motivational effects. Environments may be organized into categories such as guided meditations, nature scenes with ambient soundscapes, or avatar-led cognitive reframing sessions.

Environments are modular and may be created using industry-standard 3D content pipelines. Each module is stored locally within the device and optimized for real-time rendering using the onboard graphics processor.

### 4.2. Adaptive Selection Based on Classification and Historical Efficacy

When the biometric classification model outputs a score exceeding the therapeutic activation threshold, the system selects an appropriate environment from the library. The selection may be based on the specific state label (e.g., “acute withdrawal”) and may also consider user history, prior session outcomes, or response efficacy scores stored locally.

For example, if a prior environment successfully reduced severity scores within two minutes during similar episodes, that environment may be prioritized again. The system can also rotate or escalate intervention types if previous exposures appear ineffective, introducing novelty or changing stimulus intensity to re-engage the user.

### 4.3. Environment Features

Selected environments may include one or more of the following features. Visual modulation, such as dynamic lighting, color transitions, or visual flow patterns may influence mood or perception. Spatialized audio, including immersive soundscapes (e.g., flowing water, breathing guides, low-frequency harmonics) may regulate autonomic activity. Avatar guidance, in which a virtual agent may deliver personalized prompts, exercises, or affirmations using voice, facial expression, and gesture. The system may adapt these features in real time based on continued monitoring. For instance, if heart rate remains elevated after environment initiation, the visual tempo or voice cadence may be slowed to increase calming efficacy.

### 4.4. Timing, Escalation, and De-Escalation Criteria

Each environment is associated with timing rules that define minimum and maximum rendering durations. A typical minimum session length might be 2 minutes to allow therapeutic effects to emerge. If biometric indicators show persistent distress, the session may be extended automatically or transitioned into a more intensive scenario (e.g., guided breathing with avatar-led narration).

De-escalation logic ensures that environments do not terminate abruptly. If the system detects return to baseline biometric ranges, it may initiate a fade-out sequence or transition to a neutral resting scene before exiting the immersive mode. Conversely, if distress signals increase despite initial intervention, the system may escalate to a secondary module or prompt the user for further interaction (e.g., eye fixation, breathing sync).

### Operational Timeline

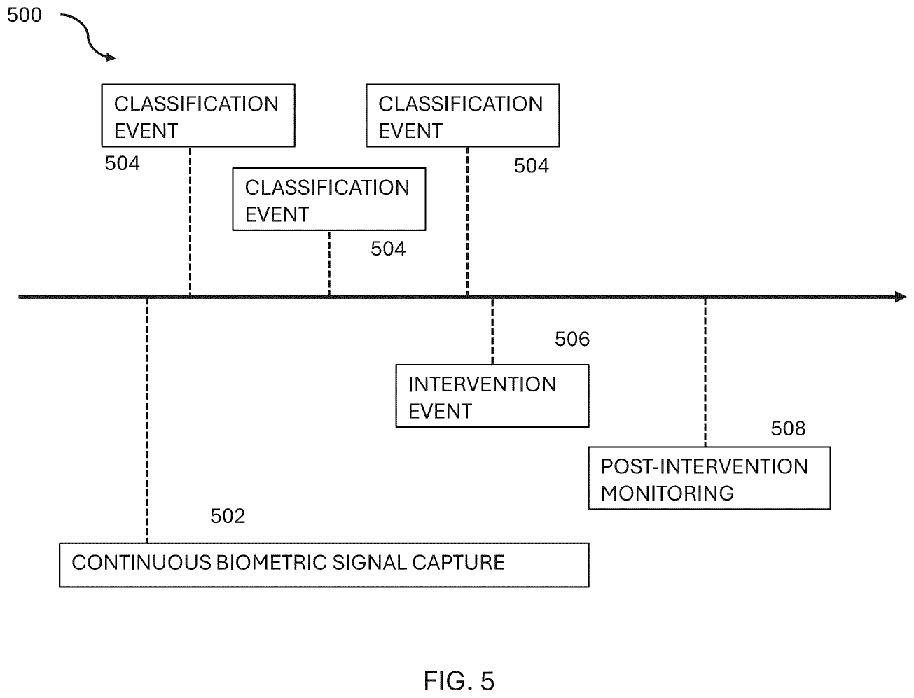

FIG. 5 illustrates a timeline 500 that represents the sequence of operations and corresponding events occurring during a sample therapeutic session utilizing the XR device. The timeline 500 is organized to depict the continuous capture of biometric signals, periodic classification intervals, and the timing of immersive intervention events.

Along the timeline 500, a continuous biometric signal capture phase 502 is shown. During this phase, physiological data from the headset's integrated biometric sensors (including heart rate, pupil dilation, and head motion) are continuously acquired and stored in rolling buffers. This phase persists throughout the session, ensuring that real-time data is available for subsequent processing. It may be appreciated that this data and these insights can further be displayed in a headworn XR device worn by a Clinician or Therapist.

At regularly spaced intervals along the timeline 500, classification events 504 are indicated. Each classification event corresponds to the application of a locally stored machine-learned model to the most recent window of preprocessed biometric data. The classification model generates an updated withdrawal severity score and, optionally, associated state labels. These intervals may be, for example, every 30 seconds, every minute, or at another predefined cadence configured in the device.

At selected points along the timeline 500, intervention events 506 are marked. Each intervention event 506 represents the automatic initiation of an immersive therapeutic environment in response to the severity score exceeding a predetermined threshold. The immersive environment, as previously described, may include adaptive visual, auditory, and avatar-guided features rendered in real time. The onset, duration, and nature of each intervention event 506 are determined by the classification output, user preferences, and historical intervention efficacy.

The timeline 500 further indicates periods 508 corresponding to sustained monitoring following an intervention. During these periods, the headset continues to acquire biometric signals and apply the classification pipeline, allowing for assessment of intervention effectiveness and determination of whether additional interventions are warranted. If biometric signals return to baseline or fall below a reset threshold, intervention rendering may be gradually de-escalated or terminated.

All signal capture intervals, classification events, and intervention actions depicted along the timeline 500 are logged in encrypted memory. This comprehensive logging facilitates trend analysis, longitudinal tracking of user progress, and optional generation of summary reports for authorized care providers.

The timeline of FIG. 5 thereby provides a structured view of the closed-loop operation of the XR device, emphasizing the continuous acquisition of biometric data, periodic state classification, and the responsive initiation of immersive therapeutic interventions throughout a session.

### Personalization and User Controls

### 5.1. User-Specific Baseline Calibration

In some embodiments, the XR device includes a calibration routine during initial setup to establish a personalized baseline for each user. During calibration, the system collects biometric signals under resting or neutral conditions and stores statistical profiles (e.g., average heart rate, typical pupil diameter, gaze fixation stability). These baseline metrics are used to normalize incoming data and improve classification accuracy by accounting for individual variation.

Over time, the system may automatically update baseline values based on longitudinal trends, daily rhythms, or environmental context. For example, if a user's baseline heart rate varies consistently with time of day, the classification model may incorporate time-weighted normalization during analysis.

### 5.2. Preferences, Thresholds, and Clinician-Set Parameters

The XR device includes a configuration interface—accessible via gaze navigation or companion device—for managing user preferences and clinician-specified parameters. Users may select preferred intervention styles (e.g., visual-only, audio-guided, avatar-led), set notification sensitivity, or enable/disable sharing of summary metrics.

For clinical use, authorized care providers may define custom severity thresholds, restrict environment categories, or adjust timing rules based on therapeutic goals. These inputs may be entered during onboarding or transmitted securely from an external management platform. The system respects role-based access control and validates any configuration changes against a consent profile.

Preferences may also be adaptive. The system may suggest changes to threshold levels or environment preferences based on intervention effectiveness, biometric recovery trends, or user feedback collected after sessions.

### 5.3. Authentication and Session Gating

To ensure secure and appropriate use, the XR device includes a biometric authentication module. This module may use facial recognition, iris scanning, or other physiological markers to verify the identity of the wearer prior to rendering any intervention. Authentication may be required once per session, or periodically depending on policy settings.

In certain modes, session gating is implemented to prevent unauthorized access to sensitive intervention content or user data. For example, if a biometric mismatch is detected during an active session, the system may suspend rendering, initiate a timeout sequence, or prompt re-authentication.

This access control layer supports secure deployment in shared or supervised environments, such as clinical settings, residential care, or mobile recovery centers.

### Secure Data Management and Optional Transmission

### 6.1. Encrypted Local Storage of Signal, Classification, and Session Data

The XR device includes a secure memory partition for storing biometric signal data, classification outputs, and metadata related to each intervention session. All stored data is encrypted at rest using hardware-based encryption mechanisms and tamper-resistant memory modules. Each data record may include timestamps, signal summaries, severity scores, selected environment identifiers, and user responses (if available).

To protect user privacy, personally identifying information (PII) is kept separate from biometric signal records unless explicit consent is provided. In typical operation, raw biometric data is retained only temporarily (e.g., for 24 to 72 hours) before being down sampled or aggregated for trend analysis, then deleted.

### 6.2. Access Logging and Tamper Detection

All access to stored data—whether by the system itself or an external authorized party—is logged in an immutable access ledger. Each log entry includes the requesting module or user identity, type of data accessed, timestamp, and outcome (e.g., granted, denied, incomplete). In addition, the headset may include tamper-detection features that trigger alerts or restrict access if unauthorized physical manipulation or memory probing is detected.

This logging infrastructure supports future compliance with privacy regulations (e.g., HIPAA, GDPR) and ensures auditability of data access and modification events. Access logs themselves are encrypted and may only be viewed via authenticated interfaces. In most embodiments, access may be controlled based on dual biometrics and/or user roles

### 6.3. Optional Transmission of Summary Metrics to Authorized Recipients

While the headset is capable of operating fully offline, it may also support secure transmission of de-identified session summaries to external systems such as electronic health record (EHR) platforms, care team dashboards, or research data vaults. Transmission is controlled via a consent profile, which governs what data types may be shared, with whom, and under what conditions.

Transmitted data is typically limited to summary statistics, such as number of sessions per day, average severity score, response latency, and intervention type. In some cases, anonymized biometric trends may be transmitted for longitudinal monitoring or remote clinician review. All outbound data is encrypted during transit using modern protocols (e.g., TLS 1.3) and signed to ensure integrity.

### 6.4. Privacy Safeguards and Consent Boundaries

The system enforces configurable privacy safeguards that respect the user's role, context, and consent history. For example, data sharing may be automatically suspended during travel, during first-time use in a new location, or when biometric trust indicators (e.g., stress levels) fall below acceptable thresholds. Users may manually override these safeguards or set permanent restrictions within their consent settings.

Consent parameters may be visualized to the user via a spatial interface within the headset, using intuitive graphics (e.g., concentric spheres or radial meters) to show what data is exposed and to whom. In some embodiments, consent changes can be made via gaze-based interaction or verbal commands, ensuring accessibility even during high-stress or low-mobility scenarios.

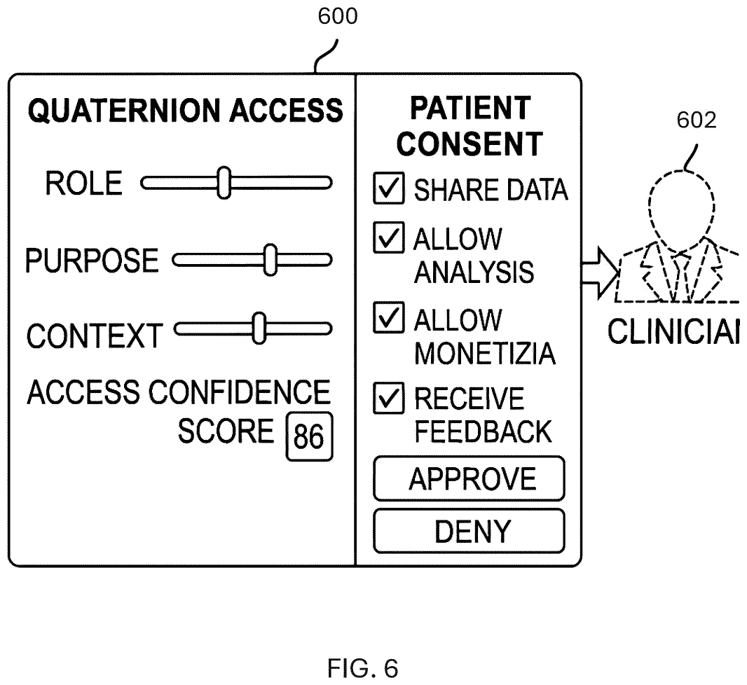

FIG. 6 illustrates a consent management interface 600 that exemplifies the spatial visualization of privacy controls and data sharing permissions within the XR device environment. The interface 600 provides an intuitive mechanism for managing complex consent parameters through visual and interactive elements rendered within the immersive display.

The quaternion access control panel on the left side of the interface employs adjustable sliders for multiple consent dimensions. The ROLE slider allows specification of which categories of healthcare professionals may access the user's data, ranging from primary care providers to specialists or emergency responders. The PURPOSE slider defines permissible uses of the data, such as direct treatment, quality improvement, or anonymized research applications. The CONTEXT slider establishes situational boundaries for data access, potentially limiting availability based on geographic location, time windows, or therapeutic phase. An ACCESS CONFIDENCE SCORE of 86 is displayed, representing the system's computed assessment of the current access request's legitimacy based on historical patterns, authentication strength, and contextual factors.

The patient consent panel on the right side presents granular control options through a checkbox interface. The SHARE DATA option enables basic transmission of de-identified session summaries to authorized recipients. The ALLOW ANALYSIS option permits deeper computational processing of biometric patterns for therapeutic optimization. The ALLOW MONETIZATION option addresses potential commercial uses of anonymized, aggregated data for product improvement or research purposes. The RECEIVE FEEDBACK option enables bidirectional communication, allowing clinicians to provide personalized guidance based on reviewed session data.

The interface includes prominent APPROVE and DENY buttons, enabling rapid consent decisions during real-time access requests. The clinician FIG. 602 represents the requesting party, with visual indicators potentially showing their credentials, institutional affiliation, and historical interaction patterns with the patient's care team.

The spatial arrangement of controls in FIG. 6 leverages the three-dimensional rendering capabilities of the XR environment, potentially allowing users to manipulate consent parameters through gaze interaction, gesture recognition, or voice commands. This design ensures that privacy management remains accessible even during periods of reduced mobility or elevated stress, maintaining user autonomy over their sensitive biometric and behavioral health data.

### 6.5 Professional Workflow Integration and Clinical Collaboration

In certain embodiments, the XR device may implement a Professional Integration Workflow Module as a software subsystem configured to support clinical workflow requirements and facilitate secure professional collaboration. The workflow module may provide multi-tier notification systems enabling automated escalation protocols for physiological states indicative of acute risk. Upon detection of a critical event, the system may trigger secure professional alerts, initiate real-time data streaming to authorized clinicians, or activate predefined intervention protocols in accordance with clinical best practices.

The workflow module may include role-based access control supporting a plurality of permission levels. Permissions may govern the extent of data and functionality available to different classes of professional users, such as emergency responders, primary clinicians, care coordinators, or technical support personnel. Access credentials may be validated through multi-factor authentication mechanisms, including biometric identification, digital certificate validation, and verification of treatment relationships.

In some aspects, the workflow integration module may support real-time collaborative review sessions in which multiple professionals access the XR device's session data through authenticated, role-appropriate interfaces. Data may be organized into tiered access layers, such that care coordinators may review session summaries and intervention frequency, clinicians may access real-time biometric trends and protocol efficacy data, and diagnostic specialists may analyze comprehensive physiological time series. Access to raw biometric data or sensitive event records may be further restricted to emergency response personnel or upon verified clinical necessity.

The system may implement secure communication protocols for inter-professional messaging, documentation of consensus decisions, and cryptographically verified collaborative annotation. Voice communication integration, structured assessment forms, and multimedia annotation support may be provided to facilitate thorough clinical documentation and collaborative care planning. All collaborative review sessions may be logged with time-stamped, cryptographically attributed interaction records to ensure transparency and regulatory compliance.

The XR device may further enable collaborative care planning and professional review of intervention sessions. Secure annotation interfaces may permit authorized professionals to attach structured notes, assessments, or protocol recommendations to specific intervention events or biometric records. Annotation metadata may be cryptographically attributed and time-stamped, and all professional interactions may be logged in an immutable audit trail to facilitate compliance with applicable privacy and regulatory standards.

In certain embodiments, the workflow module may provide secure handoff protocols, allowing transfer of session access or care responsibility between authenticated clinicians. Handoff events may require confirmation from both transferring and receiving professionals, and each event may be documented in the audit log with corresponding access context and authorization records. For urgent or emergency conditions, the system may escalate access permissions and notify additional care team members in accordance with predefined escalation policies.

Role-based access control may be implemented to support a plurality of professional permission levels. Permission categories may include, but are not limited to, emergency responders, primary clinicians, specialists, care coordinators, research collaborators, technical support, and audit reviewers. Each permission level may govern access to device features, biometric data layers, and system configuration, and may be enforced through credential validation and authenticated session management.

The professional integration module may further support automated compliance documentation and regulatory reporting functions. All professional access, annotation, intervention decisions, and audit trail records may be retained in encrypted storage and may be exported in standard formats suitable for integration with electronic health record (EHR) systems or clinical quality improvement databases. The system may generate compliance reports including access frequency analysis, intervention appropriateness metrics, and security incident summaries for regulatory validation. Anomaly detection algorithms may be applied to audit records to identify unusual access patterns or potential compliance violations, triggering automated alerts to designated compliance officers or security administrators as required.

In certain embodiments, the XR device may include an enhanced security framework incorporating dedicated hardware security modules (HSMs) for tamper-resistant credential storage, hardware-accelerated cryptographic processing, and secure authentication validation. The HSMs may provide key isolation, AES-256 encryption for biometric data at rest, RSA-4096 or elliptic curve cryptography for authentication protocols, and automated key rotation procedures that maintain forward secrecy and session integrity.

The system may further support post-quantum cryptographic algorithms to provide resilience against quantum computing threats and ensure long-term data security. Security implementations may comply with recognized standards for government-grade protection.

The security framework may include continuous authentication monitoring, combining biometric verification, behavioral pattern analysis, and anomaly detection logic. Suspicious or anomalous access attempts may automatically trigger session termination, professional notification, or escalation procedures, with forensic audit trail preservation. All access attempts, data modifications, and professional interactions may be recorded in immutable audit logs, optionally employing blockchain-based integrity protection to prevent tampering or deletion.

Automated compliance documentation and reporting may be supported, generating standardized records suitable for HIPAA, GDPR, FDA, or clinical trial requirements. Compliance reporting may include access frequency analysis, intervention appropriateness assessment, and security incident summaries, with machine learning-based anomaly detection algorithms used to identify potential compliance violations. Security administrators and compliance officers may receive automated alerts and recommended response protocols in the event of a detected anomaly.

### Multi-Tiered Data Access and Professional Collaboration

In certain embodiments, the XR device may implement a multi-tiered data access architecture to support secure and role-appropriate sharing of biometric data, therapeutic records, and session metadata in clinical environments. Access to data may be organized into a plurality of permission layers, such that each layer is tailored to the specific needs and authorization level of various professional user categories. For example, Tier 1 may provide access to behavioral metadata, session summaries, and intervention frequency data for care coordinators; Tier 2 may expose real-time biometric trends, protocol efficacy scores, and therapeutic progress indicators for direct care providers; Tier 3 may include comprehensive physiological signal analysis, temporal pattern reports, and full intervention histories for primary clinicians and specialists; and Tier 4 may provide raw physiological signals, real-time streaming data, and critical alert information for emergency response teams or intensive care staff.

The system may further include a secure, immersive data visualization engine configured to render biometric data and therapeutic insights in three-dimensional spatial interfaces. In some embodiments, this visualization engine may support collaborative review sessions, permitting multiple authenticated professionals to access synchronized data views, interact with role-appropriate information, and contribute cryptographically attributed annotations. Interactive features may include volumetric rendering of temporal biometric data, navigable 3D structures for comparative analysis, and visualization of forecasted state trajectories with confidence intervals. All collaborative review sessions and annotations may be time-stamped, attributed, and retained in a secure, immutable audit log for clinical quality improvement and compliance purposes.

Professional collaboration protocols may enable secure handoff of session access or care responsibility between clinicians, employing gesture-based or proximity-based authentication and dual-party verification. Handoff events may be confirmed by both transferring and receiving professionals and documented automatically in the audit log. Emergency protocols may provide streamlined authentication procedures for rapid access by qualified personnel, with automatic notification of location, critical parameters, recent interventions, and immediate communication with primary care providers.

In some embodiments, consultation features may enable remote, time-limited access for specialist review, with granular permission settings restricting data visibility to clinically relevant subsets. All professional collaboration, consultation, and handoff sessions may be logged and available for automated compliance reporting, ensuring transparency, traceability, and regulatory validation.

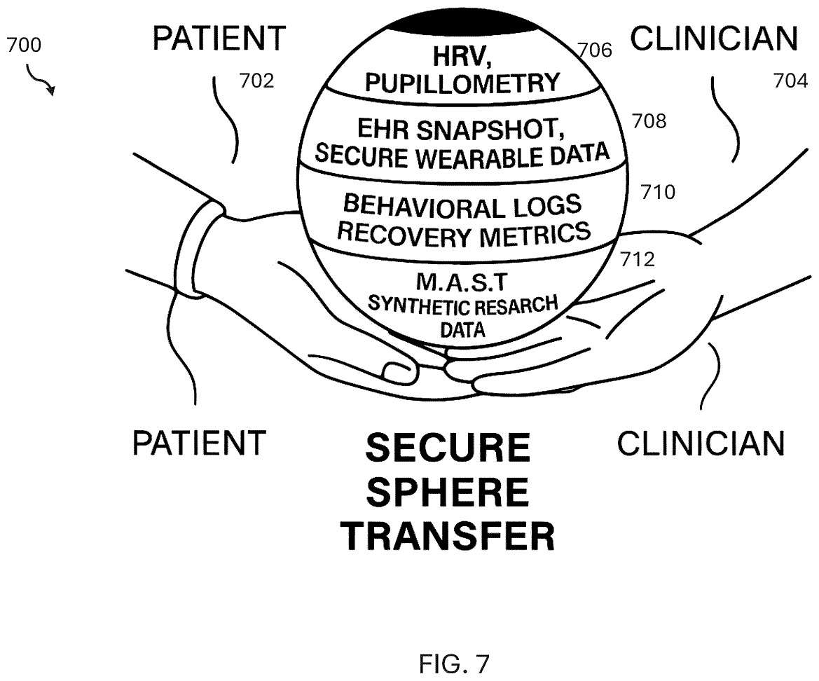

FIG. 7 illustrates a secure sphere transfer system 700 that visualizes the multi-tiered data access architecture and controlled information flow between patient 702 and clinician 704 users. The system 700 employs a spherical data containment model that organizes biometric and therapeutic information into distinct access layers, each with specific security and permission requirements.

The innermost layer 706 contains high-sensitivity biometric data including heart rate variability (HRV) measurements and pupillometry readings. These raw physiological signals represent the most granular level of patient data and require the highest level of access authorization, typically restricted to primary treating clinicians or emergency response scenarios. The data in this tier streams in real-time and provides immediate insight into autonomic nervous system activity and neurological responses.

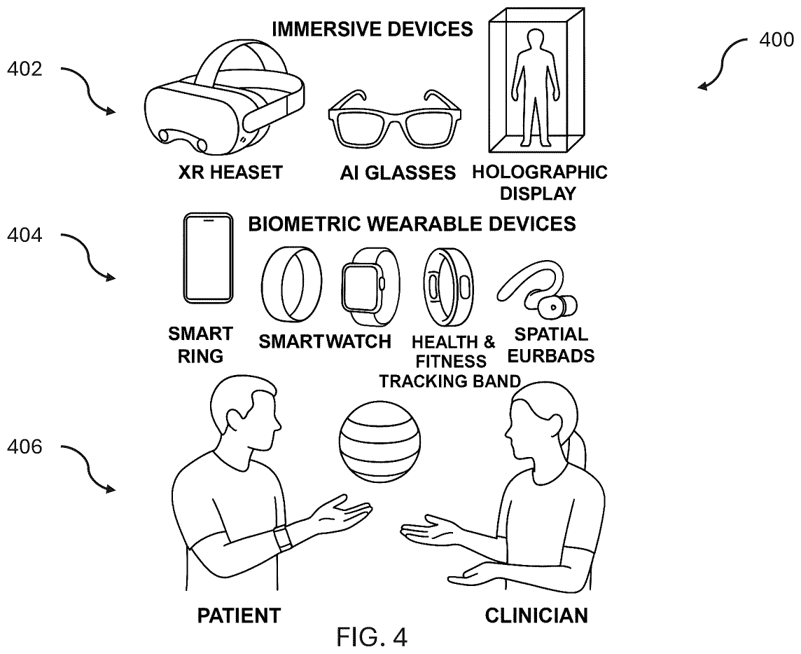

The second tier 708 encompasses electronic health record (EHR) snapshots and secure wearable device data. This layer aggregates and contextualizes the raw biometric streams into clinically interpretable formats while maintaining temporal resolution sufficient for therapeutic assessment. The EHR snapshots provide historical context and treatment continuity, while the secure wearable data incorporates measurements from the broader device ecosystem described in FIG. 4.

The third tier 710 contains behavioral logs and recovery metrics derived from intervention sessions and longitudinal monitoring. This layer represents processed therapeutic outcomes, including intervention frequency, environment effectiveness scores, and biometric recovery patterns. The behavioral logs capture user interactions with therapeutic environments without exposing raw physiological data, enabling care coordination while preserving privacy.

The outermost layer 712 comprises M.A.S.T. (Machine-Aggregated Synthetic Therapeutic) research data, representing fully anonymized and synthesized information suitable for population-level analysis and algorithm improvement. This tier supports clinical research and system optimization without compromising individual patient privacy, as the data has been transformed through differential privacy techniques and synthetic data generation.

The secure sphere transfer mechanism depicted in FIG. 7 enables authorized data movement between tiers based on the clinician's 704 role, the therapeutic context, and the patient's 702 consent settings. The spherical visualization may be rendered within the XR environment as an interactive three-dimensional object, allowing both patients and clinicians to observe data access requests, approve specific tier transfers, and monitor the scope of shared information in real-time. The spatial representation reinforces the layered security model while providing an intuitive interface for managing complex healthcare data relationships.

The professional collaboration framework may support synchronized multi-user access, permitting multiple authenticated professionals to review session data in real time, contribute structured annotations, and participate in collaborative care planning. All annotations may be cryptographically attributed to the author and time-stamped for auditability. Voice communication integration and multimedia annotation capabilities may be provided to facilitate comprehensive clinical documentation. Secure handoff protocols may require dual-party verification for transfer of care responsibility, with all actions logged for compliance.

### Multi-Device Ecosystem Integration

In certain embodiments, the XR device may be configured to operate within a multi-device biometric ecosystem that includes integration with complementary devices such as smartphones, smartwatches, smart rings, environmental monitoring systems, and professional clinical devices. The system may employ secure mesh networking protocols to facilitate low-latency communication and sub-millisecond synchronization accuracy between devices, enabling the fusion of physiological, behavioral, and contextual data from multiple sources.

Cross-device sensor fusion algorithms may be implemented to combine data streams from heterogeneous modalities, leveraging hardware-based timestamping, advanced interpolation, and statistical correlation techniques to ensure robust and accurate signal integration. In some embodiments, the XR device may be compatible with certified medical device communication protocols, supporting interoperability with clinical monitoring systems, electronic health record (EHR) platforms, and emergency response equipment.

The multi-device integration framework may include comprehensive authentication protocols, such as multi-factor verification across biometric modalities and healthcare-grade security validation, to ensure that only authorized devices and professionals can access sensitive data and system features. Fault-tolerant operation may be supported by redundant measurement capabilities and dynamic system reconfiguration, allowing the XR device to maintain therapeutic intervention functionality even during individual device failures or communication interruptions.

The system may further support automated documentation and clinical decision support, whereby aggregated biometric and environmental data can be processed to generate EHR-compatible records, protocol recommendations, and professional notifications in real time. All cross-device communications, authentication events, and data aggregation operations may be logged and retained for regulatory compliance, audit, and quality assurance purposes.

FIG. 4 illustrates a multi-device ecosystem 400 configured to support comprehensive biometric monitoring and therapeutic intervention across multiple form factors and user contexts. The ecosystem 400 depicts the integration of various device categories and their interaction with both patient and clinician users in a unified therapeutic framework.

The immersive devices category 402 comprises extended reality platforms including an XR device, AI glasses, and a holographic display. The XR device, as described in previous embodiments, serves as the primary therapeutic intervention platform with integrated biometric sensing and immersive environment rendering capabilities. The AI glasses provide a lighter-weight form factor suitable for ambulatory monitoring and discrete interventions during daily activities. The holographic display enables shared visualization experiences that may be utilized in clinical settings for collaborative review of biometric data and therapeutic progress.

The biometric wearable devices category 404 includes complementary sensing platforms that extend the system's physiological monitoring capabilities beyond the primary XR device. A smart ring provides continuous pulse oximetry and heart rate variability measurements with minimal user burden. A smartwatch offers additional biometric channels including electrodermal activity, motion tracking, and environmental context sensing. A health and fitness tracking band delivers specialized monitoring for physical activity patterns and sleep quality metrics relevant to recovery assessment. Spatial earbuds incorporate acoustic biometric sensing and may deliver audio-based interventions synchronized with the primary XR therapeutic environments.

The patient and clinician users 406 are shown interacting through the multi-device ecosystem, illustrating the bidirectional flow of biometric data, therapeutic content, and clinical insights. The patient user may wear one or more devices from categories 402 and 404 simultaneously, with the system performing sensor fusion to generate comprehensive physiological state assessments. The clinician user may access aggregated data streams, intervention histories, and real-time monitoring dashboards through authorized interfaces on any of the immersive devices 402, enabling remote supervision and collaborative care planning.

The arrangement depicted in FIG. 4 enables distributed biometric sensing with centralized analysis and intervention coordination. Each device within the ecosystem 400 may operate independently for basic monitoring functions while contributing to a unified therapeutic framework when connected. Cross-device synchronization protocols ensure temporal alignment of multimodal data streams, while role-based access controls maintain appropriate data boundaries between patient-generated physiological signals and clinician-accessible therapeutic insights.

### Example Use Cases and Therapeutic Scenarios

### 7.1. Acute Withdrawal Detection and Calming Protocol

A patient undergoing early-stage recovery wears the XR device during unsupervised periods at home. The system continuously monitors biometric signals, including heart rate, pupil dilation, and subtle head tremors. Over the course of several minutes, the classification model detects a sustained elevation in severity scores, consistent with early signs of withdrawal. Upon crossing a predefined threshold, the system automatically initiates a calming protocol consisting of a dimly lit forest environment paired with slow, spatialized ambient audio.

The patient's biometric signals begin to stabilize within three minutes, and the headset gradually fades the visual scene and transitions to a neutral resting state. The entire session is logged locally and summarized for optional review by the care team at the next appointment.

7.2. Real-Time Stress Spike Response with Guided Avatar

While navigating a public space, the user experiences an unexpected environmental trigger (e.g., entering a convenience store). The headset, operating in background monitoring mode, detects a rapid change in pupil size and head acceleration indicative of acute stress. The system delivers a low-profile, avatar-guided breathing sequence inside the headset without requiring manual interaction.

The virtual avatar appears in a peripheral zone of the field of view, offering synchronized audio prompts for slow inhalation and exhalation. The user is able to maintain social awareness while privately completing the intervention. After biometric recovery is detected, the avatar dismisses itself and the session is automatically recorded in encrypted memory. Alternatively, the Spatial Audio Positioning can include “over the shoulder” positioning further emphasizing the guidance based feeling.

### 7.3. Trend-Based Intervention Modulation Over Extended Use

Over a period of two weeks, the user engages with the XR device daily. The system tracks biometric trends and aggregates session metadata to detect longitudinal patterns. It learns that a particular category of environments (those with flowing water and low visual complexity) consistently result in faster recovery times for that user.

Using this insight, the system adaptively prioritizes water-themed interventions for future high-risk episodes. If one such episode occurs during a morning commute, the headset selects a low-intensity coastal scene with a minimal visual footprint and spatialized ocean audio. This individualized matching increases efficacy while respecting environmental context and user preference.

### 7.4. Clinician Review of De-Identified Session Metadata

In some use cases, the XR device is configured to generate periodic summary reports for authorized clinicians, enabling remote oversight of a patient's therapeutic progress. These reports are composed entirely of de-identified metadata derived from prior intervention sessions and are transmitted over a secure, encrypted connection in compliance with HIPAA and other applicable privacy regulations.

For example, on a weekly basis, the headset may compile a summary that includes the total number of interventions initiated during the reporting period, the average and maximum severity scores detected by the onboard classification model, and the median time required for the user's biometric signals to return to baseline after each intervention. The report may also contain a breakdown of which therapeutic environments were most frequently used and how effective each was in reducing biometric distress indicators (such as heart rate, gaze instability, or head movement variance) within a predefined recovery window.

The purpose of these summaries is not to relay raw biometric signals, but rather to provide high-level, actionable insights that support clinician decision-making. The information may be visualized in a dashboard or integrated with the patient's broader treatment plan, allowing the clinician to track trends over time, identify patterns of risk escalation, and adjust care protocols accordingly. For example, if the data indicate repeated evening interventions with prolonged recovery times, the clinician might recommend changes in medication timing, behavioral strategies, or scheduling of support resources.

Importantly, the patient retains full control over what information is shared and with whom. The consent framework integrated into the headset allows the user to toggle clinician access on or off at any time, and to view a real-time summary of what categories of data are being transmitted. Access settings may also be customized to allow one-time reports, recurring summaries, or time-limited access, ensuring that data sharing always aligns with the user's preferences and therapeutic context.

As will be appreciated by one skilled in the art, aspects of the present invention may be embodied as a system, method, or computer program product, and may be implemented in hardware, software, or any suitable combination thereof. Embodiments may include code executed by one or more processors to perform the functions described herein.

The invention may be embodied in a non-transitory computer-readable medium storing instructions that, when executed by a processor, cause a computing device to perform one or more of the steps described herein. Suitable storage media include RAM, ROM, flash memory, magnetic drives, optical media, or other forms of tangible memory. The invention may also be embodied in software as a service (Saas), edge computing configurations, or cloud-based systems with multitenant support.

The system may be deployed on a standalone XR device or in distributed form across multiple devices interconnected via wireless or wired communication links, including the Internet. In some embodiments, client devices may interact with back-end servers through application programming interfaces (APIs), and data may be stored, synchronized, or analyzed using private or public cloud infrastructure.

Machine learning techniques may be used to perform classification, prediction, or recommendation functions described herein. These techniques may include supervised or unsupervised learning models such as neural networks, decision trees, clustering algorithms, or deep learning architectures. ML models may be trained on historical biometric or behavioral data, and may be updated over time to improve predictive accuracy or personalization. In certain embodiments, ML models are executed on-device for real-time inference, optionally accelerated using dedicated hardware.

Where applicable, flowcharts, block diagrams, and signal-processing pipelines described in this document may be implemented as hardware modules, software modules, or hybrid logic executed by general-purpose processors, digital signal processors (DSPs), or microcontrollers. System components described as “modules” or “engines” may be implemented using discrete or integrated logic, object-oriented code, functional blocks, or combinations thereof.

Unless otherwise specified, the singular terms “a,” “an,” and “the” are meant to include plural forms, and the term “comprising” does not exclude additional elements or steps. The invention is not limited to the specific embodiments disclosed herein, but rather extends to all equivalents consistent with the claims.

1. An extended reality (XR) device operable for real-time biometric intervention for substance use disorder treatment, the headset comprising:
a head-mounted display operable to render immersive therapeutic environments;
one or more biometric sensors integrated within the headset and operable to detect real-time physiological signals including at least two of: pupil diameter, eye fixation duration, heart rate, and head movement;
a memory storing a machine-learned classification model trained to associate physiological signal patterns with at least one cognitive or affective state indicative of withdrawal severity or relapse risk;
a processor operatively coupled to the biometric sensors and the head-mounted display, the processor operable to:
collect physiological signals from the biometric sensors over a rolling time window;
preprocess the signals to remove noise and normalize sampling rates;
apply the classification model to the preprocessed signals to generate a withdrawal severity score;
compare the score to a predetermined threshold; and
upon determining the threshold is exceeded, initiate rendering of a corresponding immersive therapeutic environment on the head-mounted display that is selected to mitigate a detected state,
wherein the therapeutic environment comprises at least one of: spatialized audio, guided avatar interaction, or ambient visual modulation, selected based on the classification output.

2. The XR device of claim 1, wherein the biometric sensors include at least one infrared camera operable for pupil tracking.

3. The XR device of claim 1, wherein the biometric sensors include a photoplethysmography (PPG) module embedded within the headset housing.

4. The XR device of claim 1, wherein the processor is further operable to store the collected physiological signals and the preprocessed physiological signals in a local circular buffer for temporal analysis.

5. The XR device of claim 1, wherein the machine-learned classification model is a neural network trained on a labeled dataset of biometric signal profiles associated with withdrawal episodes and successful recoveries.

6. The XR device of claim 1, wherein the processor is operable to apply a temporal smoothing function across at least three classification outputs before initiating a therapeutic environment.

7. The XR device of claim 1, wherein the guided avatar interaction includes verbal prompts synchronized with real-time gaze tracking.

8. The XR device of claim 1, wherein the immersive therapeutic environment is rendered for a minimum duration of 90 seconds unless the biometric signals indicate elevated distress.

9. The XR device of claim 1, further comprising a biometric authentication module operable to verify user identity prior to rendering a therapeutic environment.

10. The XR device of claim 1, wherein the headset includes a wireless communication interface operable to transmit summary biometric metrics to a server upon completion of a therapeutic session.

11. A method for real-time biometric intervention using an extended reality (XR) device, the method comprising:
continuously acquiring physiological signals from one or more integrated biometric sensors within the XR device, the signals including at least two of: pupil diameter, eye fixation duration, heart rate, and head movement;
preprocessing the physiological signals to remove noise and normalize sampling rates;
aggregating the preprocessed signals from multiple modalities into a feature vector;
providing the feature vector to a locally stored machine-learned classification model;
generating a withdrawal severity score using the classification model;
comparing the withdrawal severity score to a predetermined activation threshold;
in response to the withdrawal severity score exceeding the activation threshold, triggering an intervention by selecting and rendering an immersive therapeutic environment through the XR device; and
logging results of the classification and any triggered interventions in local memory.

12. The method of claim 11, wherein acquiring physiological signals includes capturing pupil tracking data using at least one infrared camera.

13. The method of claim 11, wherein acquiring physiological signals includes detecting pulse waveform data using a photoplethysmography (PPG) module.

14. The method of claim 11, further comprising storing the collected physiological signals and the preprocessed physiological signals in a circular buffer for temporal analysis.

15. The method of claim 11, wherein the machine-learned classification model is a neural network trained on labeled biometric signal profiles associated with withdrawal episodes.

16. The method of claim 11, further comprising applying a temporal smoothing function across at least three classification outputs before triggering the intervention.

17. The method of claim 11, wherein rendering the immersive therapeutic environment includes presenting guided avatar interactions with verbal prompts synchronized to real-time gaze tracking.

18. The method of claim 11, wherein the immersive therapeutic environment is rendered for a minimum duration of 90 seconds unless the biometric signals indicate elevated distress.

19. The method of claim 11, further comprising verifying user identity through biometric authentication prior to rendering the therapeutic environment.

20. The method of claim 11, further comprising transmitting summary biometric metrics to a server via a wireless communication interface upon completion of a therapeutic session.
